(19) United States
(12) Patent Application Publication
(10) Pub. No.: US 2018/0113120 A1
(43) Pub. Date: Apr. 26, 2018

(54) GENOTOXICITY ASSAY WITH CYTOPLASM BOUNDARY LABELING

(71) Applicants: Christopher James CAPACCIO, Baltimore, MD (US); Richard Joseph KOWALSKI, JR., Leesburg, VA (US)

(72) Inventors: Christopher James CAPACCIO, Baltimore, MD (US); Richard Joseph KOWALSKI, JR., Leesburg, VA (US)

(73) Assignee: NORTHROP GRUMMAN SYSTEMS CORPORATION, Falls Church, VA (US)

(21) Appl. No.: 15/333,604

(22) Filed: Oct. 25, 2016

(51) Int. Cl. G01N 33/50; C12Q 1/02; G01N 35/00; G01N 33/58

(52) U.S. Cl. CPC G01N 33/5091; C12Q 1/02; G01N 2333/37; G01N 33/582; G01N 2333/4724; G01N 35/00029

(57) ABSTRACT

A method of detecting chromosomal damage in a subject is described that includes the steps of stimulating proliferation of the lymphocytes of a blood sample from a subject by contacting them with a labeled mitogen; growing the stimulated lymphocytes in cell culture; contacting the lymphocytes with a mitotic arrest agent to arrest the lymphocytes as binucleate lymphocytes; staining the lymphocytes with a DNA binding dye; detecting labeled mitogen to define the cytoplasm boundary of binucleated lymphocytes; counting the micronuclei in one or more binucleated lymphocytes to obtain an observed micronuclei value; and determining the level of chromosomal damage in the subject by comparing the observed micronuclei value with a control micronuclei value. The method can be used to conduct biological dosimetry on subjects who are suspected of having been exposed to radiation.

### STATEMENT REGARDING FEDERALLY SPONSORED RESEARCH

This invention was made with government support under Government Contract Number HHS0100201000002C for “Biodosimetry After Radiologic and Nuclear Events.” The Government has certain rights in this invention.

### BACKGROUND

It is important to be able to determine if a subject has been exposed to cytotoxic and genotoxic agents. This exposure may arise from fissile material, radiologic emergencies, and other types of radiation. Examples can arise in people who work with, or live near, nuclear material; the atomic bomb survivors of Hiroshima and Nagasaki; Chernobyl cleanup workers; those living in the community of the Goiania accident; amongst others. Since the mid-1960's, biological dosimetry has been an important analysis technique to determine the extent of radiation exposure. Historically, the biomarker assayed for biological dosimetry was dicentric chromosomes, chromosomes with two or more centromeres, in blood lymphocytes. “Cytogenetic Dosimetry: Applications in Preparedness for and Response to Radiation Emergencies,” EPR-Biodosimetry 2011, International Atomic Energy Agency Publications (IAEA). A strong correlation between results obtained in vivo to those obtained in vitro established a dose-effect relationship sufficient to create a dicentric chromosomes calibration curve by irradiating blood samples. This calibration curve enables the quantification of the amount of radiation to which someone has been exposed. This is further supported by the observation that about 0.5-1.0 per 1,000 cells with dicentric chromosomes are observed in the normal population.

Biological dosimetry has grown beyond the initial dicentric assay to now include various other dosimetry assays such as Premature Chromosome Condensation (PCC), metaphase spread Dicentric (and ring) Chromosome Assay (DCA), Florescent in situ Hybridization (FISH) metaphase translocation assay, and the Cytokinesis-block Micronucleus (CBMN) assay. The CBMN assay is the preferred method for measuring micronuclei in cultured cells, and can detect apoptosis, dicentric chromosomes, chromosome breakage and loss, gene amplification, and necrosis, all of which may arise from cytotoxic and genotoxic events at any point during a cells life cycle. Fenech M., Nat Protoc., 2(5):1084-104 (2007).

CBMN has become the preferred method to detect micronuclei, a biomarker of chromosome breakage, in binuclear cells. The assay works by applying a cytokinesis blocking agent, cytochalasin-B (Cyt-B), which arrests cytokinesis so dividing cells remain binucleated. Cyt-B deploymerizes the actin filament ring required for cytokinesis to create the cleavage furrow necessary to form two daughter cells. MacLean-Fletcher S. and Pollard T. D., Cell 20, 329 (1980). Peripheral lymphocyte populations used for this assay usually reside in the GO stage of the cell cycle. Therefore, mitogens are used to coax isolated peripheral lymphocytes into the cell cycle. Examples of mitogens that can be used include phytohaemagglutinin (PHA), which has at least four variants, types E, L, M and P, and concanavalin A (ConA).

The CBMN assay may use a 10% Giemsa stain in a potassium phosphate buffer or a Diff-Quik stain, a Giemsa stain variant, for light microscopy. Fenech M., Nat Protoc., 2(5):1084-104 (2007). For fluorescent microscopy, it may use acridine orange in phosphate-buffered saline, a DNA intercalator. Another fluorescent imaging technique used in CBMN assays is a 4′,6′-diamidino-2-phenylindole (DAPI) stain, another DNA probe. However, these staining methods do not stain the cytoplasm in a specific fashion, and brief field stains that allow cytoplasm detection have very high non-specific detection rates. Therefore, there is a need to improve binuclear visualization, lower the production false positive production rate, and improve the sensitivity of the CBMN assay.

### SUMMARY

The inventors have developed a variation of the CBMN assay under the trade name of CytoRADx. This technology couples a label (e.g., AlexaFluor-488) to a mitogen (e.g., ConA), which is a lymphocyte mitotic inducer. Lymphocytes contain cell surface receptors that irreversibly bind to mitogen molecules. Therefore, labeled mitogen allows the cell's perimeter to be visualized. An additional chemical added later in culture arrests the cells in cytokinesis. Together, when these two agents are used with a DNA binding stain, such as DAPI, the cell's chromosomes can be assess for chromosomal damage.

Accordingly, in one aspect, the invention provides a method of detecting chromosomal damage in a subject that includes the steps of stimulating proliferation of the lymphocytes of a blood sample from a subject by contacting them with a labeled mitogen; growing the stimulated lymphocytes in cell culture; contacting the lymphocytes with a mitotic arrest agent to arrest the lymphocytes as binucleate lymphocytes; staining the lymphocytes with a DNA binding dye; detecting labeled mitogen to define the cytoplasm boundary of binucleated lymphocytes; counting the micronuclei in one or more binucleated lymphocytes to obtain an observed micronuclei value; and determining the level of chromosomal damage in the subject by comparing the observed micronuclei value with a control micronuclei value.

In some embodiments, the method includes the step of obtaining a blood sample from the subject. In other embodiments, the method includes the step of isolating lymphocytes from the blood sample before contacting them with a labeled mitogen. In further embodiments, the subject is suspected of having been exposed to a hazardous level of radiation. In yet further embodiments, the method includes the step of fixing the lymphocytes to a substrate before staining the lymphocytes. In additional embodiments, blood samples from a plurality of subjects are evaluated using the method, while in further embodiments, one or more of the steps are automated and/or uses an algorithm.

### BRIEF DESCRIPTION OF THE FIGURES

The accompanying drawings, which are incorporated in and constitute a part of this specification, illustrate some embodiments disclosed herein, and together with the description, serve to explain principles of the disclosed embodiments.

FIG. 1 provides a schematic representation of the method of detecting chromosomal damage in a subject.

FIG. 2 provides a schematic representation of a cell that has been arrested in an early stage of cytokinesis, showing a variety of small molecules that can be used to dissect cytokinesis regulation in time and space. Key cytokinesis proteins and their corresponding small molecule inhibitors (e.g., cytochalasin B) are shown.

FIG. 3 provides a representation of binucleated lymphocytes having micronuclei (MN) or a nucleoplasmic bridge (NPB).

### DETAILED DESCRIPTION

The present invention provides a method of detecting chromosomal damage in a subject. The method includes the steps of stimulating proliferation of the lymphocytes of a blood sample from a subject by contacting them with a labeled mitogen; growing the stimulated lymphocytes in cell culture; contacting the lymphocytes with a mitotic arrest agent to arrest the lymphocytes as binucleate lymphocytes; staining the lymphocytes with a DNA binding dye; detecting labeled mitogen to define the cytoplasm boundary of binucleated lymphocytes; counting the micronuclei in one or more binucleated lymphocytes to obtain an observed micronuclei value; and determining the level of chromosomal damage in the subject by comparing the observed micronuclei value with a control micronuclei value. The method can be used to conduct biological dosimetry on subjects who are suspected of having been exposed to radiation.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a sample” includes a plurality of such samples.

The terms “subject,” and “patient” are used interchangeably herein, and generally refer to a mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos. Evaluation of humans is of particular interest.

### Method of Detecting Chromosomal Damage

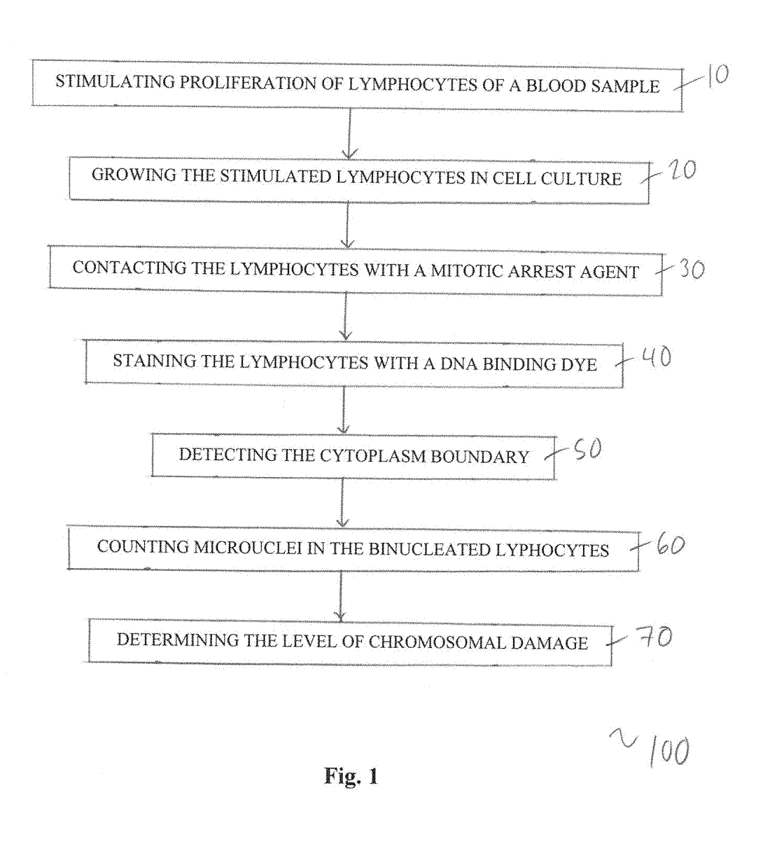

One aspect of the invention provides a method of detecting chromosomal damage 100 in a subject. A schematic representation of the overall steps involved in this method is provided by FIG. 1. The method includes the steps of: a) stimulating proliferation of the lymphocytes of a blood sample from a subject by contacting them with a labeled mitogen 10; b) growing the stimulated lymphocytes in cell culture 20; c) contacting the lymphocytes with a mitotic arrest agent to arrest the lymphocytes as binucleate lymphocytes 30; d) staining the lymphocytes with a DNA binding dye 40; e) detecting labeled mitogen to define the cytoplasm boundary of binucleated lymphocytes 50; f) counting the micronuclei in one or more binucleated lymphocytes to obtain an observed micronuclei value 60; and g) determining the level of chromosomal damage in the subject by comparing the observed micronuclei value with a control micronuclei value 70. Note that the steps of the method of detecting chromosomal damage can be carried out in the order described, but in some embodiments the sequence of one or more of the steps are varied. For example, in some embodiments, the micronuclei can be counted (step f) before the labeled mitogen is detected to define the cytoplasm boundary of binucleated lymphocytes.

The present invention provides a modified version of the cytokinesis-block micronucleus (CBMN) assay, in which labeled mitogen is used to mark the cells to facilitate analysis of the cells for micronuclei and other features indicating chromosomal damage. A protocol for the CBMN assay is described by Fenech (Fenech, Nature Protocols, 2, 1084-1104 (2007)), and is incorporated herein by reference. A protocol for the CBMN assay is also provided in the Example herein.

Chromosomal damage can result from exposure to radiation or genotoxic chemicals. Other factors that can cause chromosomal damage include lifestyle factor such as diet, various medical therapies, and even increased exposure to ultraviolet radiation due to atmospheric ozone depletion. Because the present method is capable of detecting chromosomal damage, it can be used to identify individuals who have been exposed to radiation or genotoxic chemicals, identify individuals who have increased sensitivity to chromosomal damage, screen new chemicals for their genotoxic effects, determine the level of chromosomal damage in a population following a major accident, or routinely monitor individuals who are occupationally exposed to agents or radiation that can cause chromosomal damage. In some embodiments, the subject is suspected of having been exposed to a genotoxic chemical, while in other embodiments the subject is suspected of having been exposed to a hazardous level of radiation. A subject may be suspected of having been exposed to radiation or a genotoxic chemical as a result of displaying other symptoms, or as a result of the subject having been present in an area of known exposure to radiation or a genotoxic chemical.

The level of exposure to radiation or a genotoxic chemical may be used to guide subsequence treatment of the subject. For example, if the assay indicates the subject has been exposed to 2 or fewer gray (Gy), no treatment is necessary, whereas exposure to from about 2 to 4 Gy suggests treatment with IV fluids, exposure from about 4 to 6 Gy suggests treatment with medical countermeasures for acute radiation syndrome such as administration of G-CSF or GM-CSF, and exposure to about 6 to about 8 Gy suggests treatment with bone marrow transplant.

Blood samples for use with the subject methods may be obtained from a subject by any suitable method, including but not limited to withdrawing blood from a subject using a needle and syringe. In some embodiments, blood samples may be collected from a subject using a finger-stick technique, wherein the subject's skin is punctured and a sufficient amount of blood is “milked” or expressed from the puncture. Blood samples suitable for use in the subject methods include both venous and arterial blood samples. Any blood sample collection procedure may be readily adapted for use with the subject methods. Typically a small sample (e.g., 0.01 to 10 mL; 0.05 to 5 mL; or 0.5 to 1 mL) of blood is all that is needed to carry out the method of the invention, with 50 μL being commonly used.

An anticoagulant such as lithium heparin may be included to prevent coagulation of the blood sample. The blood sample may be fresh or stored. Blood samples may be or have been stored or banked under suitable tissue storage conditions. For example, the blood can maintained at a temperature range of about 6 to about 22° C. If the blood sample is stored, it is preferable that it not be stored more than 72 hours. The blood sample may be a tissue sample expressly obtained for the assays of this invention or a tissue sample obtained for another purpose which can be subsampled for the assays of this invention. Blood samples may also be chilled or frozen shortly after collection if they are being stored to prevent deterioration of the sample.

The sample may be pretreated as necessary by dilution in an appropriate buffer solution, heparinized, concentrated if desired, or fractionated by any number of methods including but not limited to ultracentrifugation, fractionation by fast performance liquid chromatography (FPLC) or HPLC, or precipitation of apolipoprotein B containing proteins with dextran sulfate or other methods. Any of a number of standard aqueous buffer solutions at physiological pH, such as phosphate, Tris, or the like, can be used.

In some embodiments, whole blood is used to carry out the analysis. In other embodiments, once a blood sample from one or more subjects has been provided, lymphocytes are isolated from the blood sample(s). Lymphocytes found in the blood are typically referred to as peripheral blood lymphocytes. Peripheral blood lymphocytes (PBL) are mature lymphocytes that circulate in the blood, and include T cells, natural killer (NK) cells and B cells. Lymphocytes can be identified by their large nucleus, which facilitates analysis of the chromosomes, and can be stimulated to proliferate, unlike erythrocytes which lack a cell nucleus.

Isolating lymphocytes, as used herein, does not refer to a complete removal of other cells and/or material, but rather represents a partial purification of the lymphocytes. It is helpful to isolate the lymphocytes from other cells such as erythrocytes in order to facilitate analysis and culturing of the lymphocytes. A variety of methods are known to those skilled in the art for isolating lymphocytes from other blood components. For example, one method for isolating lymphocytes is the use of density gradient centrifugation using Ficoll-Paque® centrifugation media. Ficoll-Paque® is a sterile, density media containing Ficoll PM400, sodium diatrizoate and disodium calcium EDTA, with a density that has been optimized for the isolation of human lymphocytes from peripheral blood. To isolate lymphocytes from blood using this method, the blood is typically diluted using buffer, mixed with Ficoll-Paque® media, and then centrifuged in tubes at 400 g for about 30 minutes. The upper lymphocyte layer is then removed from the tubes, diluted with Hank's Balanced Salt Solution (HBSS), and centrifuged again at 100 g for 10 minutes to obtain a cell pellet of lymphocytes, which is then resuspended in suitable media. Any residual erythrocytes can be lysed using a lytic reagent such as Zap-Oglobin®.

The isolated lymphocytes are then resuspended in culture medium, unless whole blood is used for the analysis, in which case it can be analyzed directly. In some embodiments, the cells are re-suspended at a density of about 1×10⁶ cells per mL. The lymphocytes are then stimulated to proliferate by contacting them with a labeled mitogen. The labeled mitogen remains associated with the lymphocytes, and can be used to define the cytoplasm boundary of the lymphocytes to allow visualization for manual or automated analysis. Labeled mitogen is mitogen that includes a dye or other compound that allows for detection of the mitogen. For example, dyes may be used to allow visual or fluorescent detection of the labeled mitogen.

Mitogens are chemical agents that trigger mitosis in cells such as lymphocytes. Examples of mitogens include phytohaemogglutinin (PHA), concanavalin A (ConA), lipopolysaccharide (LPS), and pokeweed mitogen (PWM). The amount of mitogen that should be added to stimulate the cells is known to those skilled in the art. For example, a concentration of about 30 μg/mL of PHA can be provided to lymphocytes in cell culture to stimulate the lymphocytes.

The mitogen is modified to carry a label (i.e., an imaging compound). ConA bearing AlexaFluor-488 is commercially available, as is ConA labeled with the green fluorescent dye CF488A (from Biotium™). Examples of labels include visually detected compounds such as optical imaging agents and fluorescent compounds. Examples of suitable fluorescent compounds that can be used include, but are not limited to, fluorescein (FITC), 5-carboxyfluorescein-N-hydroxysuccinimide ester, 5,6-carboxymethyl fluorescein, nitrobenz-2-oxa-1,3-diazol-4-yl (NBD), fluorescamine, OPA, NDA, indocyanine green dye, the cyanine dyes (e.g., Cy3, Cy3.5, Cy5, Cy5.5 and Cy7), 4-acetamido-4′-isothiocyanatostilbene-2,2′ disulfonic acid, acridine, acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate, N-(4-anilino-1-naphthyl)maleimide, anthranilamide, BODIPY, Brilliant Yellow, coumarin, 7-amino-4-methylcoumarin (AMC, Coumarin 120), 7-amino-4-trifluoromethylcoumarin (Coumaran 151), cyanosine, 4′,6-diaminidino-2-phenylindole (DAPI), 5′,5″-dibromopyrogallol-sulfonaphthalein (Bromopyrogallol Red), 7-diethylamino-3-(4′-isothiocyanatophenyl)-4-methylcoumarin diethylenetriamine pentaacetate, 4,4′-diisothiocyanatodihydro-stilbene-2,2′-disulfonic acid, 4,4′-diisothiocyanatostilbene-2,2′-disulfonic acid, 5-[dimethylamino]naphthalene-1-sulfonyl chloride (DNS, dansylchloride), 4-(4′-dimethylaminophenylazo)benzoic acid (DABCYL), 4-dimethylaminophenylazophenyl-4′-isothiocyanate (DABITC), eosin, eosin isothiocyanate, erythrosin B, erythrosine, isothiocyanate, ethidium bromide, ethidium, 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein isothiocyanate, IR144, IR1446, Malachite Green isothiocyanate, 4-methylumbelliferone, ortho cresolphthalein, nitrotyrosine, pararosaniline, Phenol Red, B-phycoerythrin, o-phthaldialdehyde, pyrene, pyrene butyrate, succinimidyl 1-pyrene butyrate, Cibacron Blue 3B-A, 6-carboxy-X-rhodamine (ROX), 6-carboxyrhodamine (R6G), lissamine rhodamine B sulfonyl chloride rhodamine (Rhod), 5,6-tetramethyl rhodamine, rhodamine B, rhodamine 123, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (6-TAMRA), tetramethyl rhodamine, tetramethyl rhodamine isothiocyanate (TRITC), riboflavin, rosolic acid, coumarin-6, and the like, including combinations thereof.

In some embodiments, the label is an optical imaging agent such as a Wright-Giemsa staining reagent. Wright-Giemsa staining reagents generally comprise varying amounts of both Wright's stain and Giemsa stain compounds, which both comprise thiazin and eosin dyes. The thiazin and eosin dyes in a Wright-Giemsa staining reagent bind to and color various components of biological samples so that they can be visualized under microscopic examination. Thiazin dyes generally include, but are not limited to, methylene blue, methylene violet, azure A, azure B, azure C, and thionin. Thiazin dyes also include oxidation products of these compounds. Eosin dyes generally include, but are not limited to, eosin Y and eosin B. Wright-Giemsa staining reagents and staining stock solutions comprising various amounts of thiazin and eosin compounds are commercially available to the public from a variety of sources and suppliers.

In general, a label can be conjugated to the mitogen by any suitable technique. The labels(s) can be coupled to a mitogen either directly or indirectly (e.g. via a linker group). In some embodiments, the label is directly attached to a functional group capable of reacting with the label. For example, some mitogens include amino acids such as lysine that have a free amino group that can be capable of reacting with a carbonyl-containing group, such as an anhydride or an acid halide, or with an alkyl group containing a good leaving group (e.g., a halide). Proteins also contain glutamic and aspartic acids. The carboxylate groups of carbohydrates or amino acids also present attractive targets for functionalization using carbodiimide activated linker molecules; cysteines can also be present which facilitate chemical coupling via thiol-selective chemistry (e.g., maleimide-activated compounds). Proteins can also contain tyrosines, which can be modified using diazonium coupling reactions. See Hermanson, G. T., Bioconjugation Techniques. (Academic Press, 2008) and Pokorski, J. K. and N. F. Steinmetz, Mol Pharm 8(1): 29-43 (2011), the disclosures of which are incorporated herein by reference.

Alternatively, a suitable chemical linker group can be used. A linker group can serve to increase the chemical reactivity of a substituent on either the label or the mitogen, and thus increase the coupling efficiency. Suitable linkage chemistries include maleimidyl linkers, which can be used to link to thiol groups, isothiocyanate and succinimidyl (e.g., N-hydroxysuccinimidyl (NHS)) linkers, which can link to free amine groups, diazonium which can be used to link to phenol, and amines, which can be used to link with free acids such as carboxylate groups using carbodiimide activation. Useful functional groups are present on mitogens based on the particular amino acids present, and additional groups can be designed into recombinant mitogen proteins. It will be evident to those skilled in the art that a variety of bifunctional or polyfunctional reagents, both homo- and hetero-functional (such as those described in the catalog of the Pierce Chemical Co., Rockford, Ill.), can be employed as a linker group. Coupling can be affected, for example, through amino groups, carboxyl groups, sulfhydryl groups or oxidized carbohydrate residues.

After the lymphocytes have been stimulated with the labeled mitogen, they are given time to grow in cell culture and are then contacted with a mitotic arrest agent to arrest the division of the lymphocytes at the correct stage to provide binucleate lymphocytes. Growth conditions for lymphocytes are known to those skilled in the art. For example, lymphocytes can be grown at 37° C. in a humidified atmosphere containing 5% CO₂. The cells should be grown for a time period that results in a large number of lymphocytes being in binucleate form when contacted with the mitotic arrest agent. For example, when the mitogen PHA is used and cells are grown at 37° C. in a humidified atmosphere containing 5% CO₂, the lymphocytes should be contacted with the mitotic arrest agent at 44 hours after stimulation with the labeled PHA. In other embodiments, the cells should be contacted with the mitotic arrest agent within 40 to 50 hours after stimulation with the labeled mitogen.

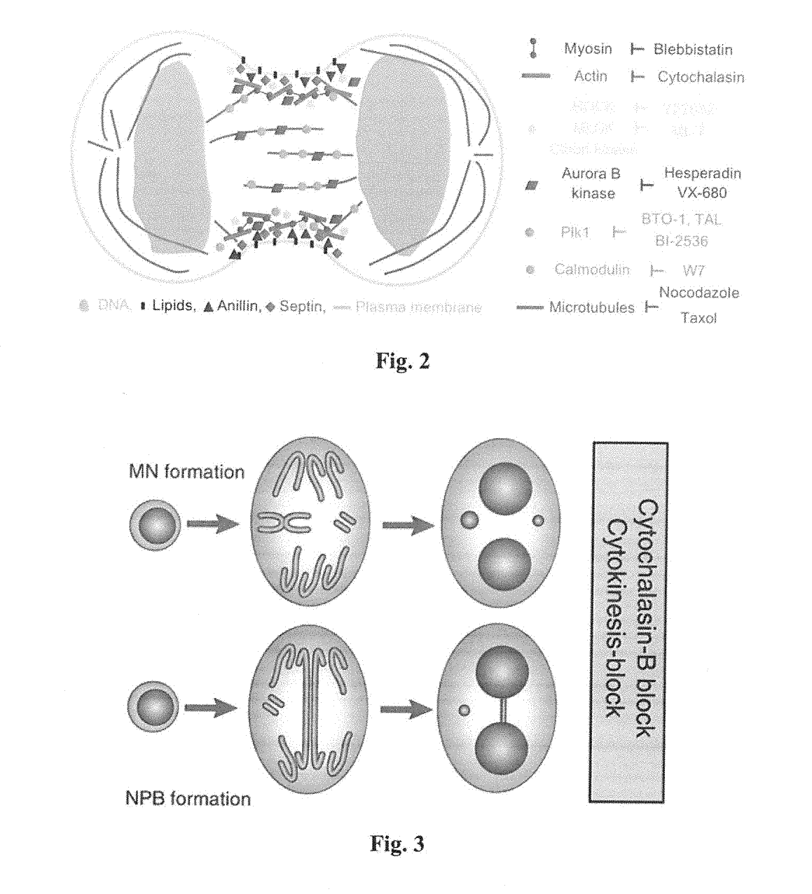

The cell cycle is a series of cellular events that allows a single cell to become two through a process that duplicates a parent cell's DNA and divides that DNA equally amongst two daughter cells. The cell cycle has three major stages: interphase, mitosis, and cytokinesis. Cytokinesis immediately follows the telophase. While there is still one cell in the telophase, this cell is binucleated, it contains double the number of chromatids of the parent cell, 92 (for human cells), rather than 46. For the daughter cells to become mononucleated, a contractile ring assembled during cytokinesis physically pinches the binucleated cell in two. Composed of actin proteins, the contractile ring creates a cleavage furrow during the physical isolation of a binucleated cell into two daughter cells. FIG. 2 shows a binucleate cell in which cytochalasin B has inhibited actin polymerization, thereby halting division of the bincucleated lymphocyte.

A variety of mitotic arrest agents are known to those skilled in the art. Mitotic arrest agents are cytoskeletal drugs that target the microtubules to prevent polymerization. Examples of mitotic arrest agents include colchicine, democolcine, cytochalasins (e.g, cytochalasin B and cytochalasin D), nocodazole, and vinblastine. In some embodiments of the invention, the mitotic arrest agent is cytochalasin D. See Atilla-Gokcumen G. E., et al., ACS Chem. Biol. 5, 79 (2010).

Once the lymphocytes have been arrested in the in their binucleated form, they can be stained with a DNA binding dye. The lymphocytes are typically allowed to remain in cell culture for a certain amount of time after exposure to the mitotic arrest agent before being harvested and stained. In some embodiments, the lymphocytes remain in culture for 20 to 30 hours after being arrested before being harvested for staining with a DNA binding dye, while in other embodiments the lymphocytes remain in culture for 24 to 28 hours after being arrested before being harvested.

A variety of DNA binding dyes are known to those skilled in the art. Non-specific double-stranded DNA detection and visualization can be monitored using intercalating or minor groove-binding fluorophores, such as 4′,6-diamidino-2-phenylindole (DAPI), Hoechst 33258, DRAQ5, and others. Martin et al., Cytometry Part A, 67A, 45-52 (2005). In some embodiments, the DNA binding dye is DAPI. For a review of various dyes used for nucleic acid visualization, see Boutorine et al., Molecules. 18(12):15357-97 (2013), the disclosure of which is incorporated herein by reference. Use of the DNA binding dye allows the nuclei and micronuclei in the cell to be visualized using methods such as fluorescence microscopy. In some embodiments, the DNA binding dye and the label fluoresce at the same frequency, while in other embodiments the DNA binding dye and the label fluoresce at different frequencies.

In some embodiments, the method also includes the step of fixing the lymphocytes to a substrate before staining the lymphocytes. Lymphocytes can be fixed to a substrate by contacting the lymphocytes with a fixation reagent. Suitable fixation reagents generally include organic solvents, such as alcohols or acetone, which remove lipids and dehydrate cells. Exemplary fixation reagents include methanol, ethanol, acetone, solutions comprising these reagents, and mixtures thereof. In some embodiments, a solution of pure methanol is used as a fixation reagent. In some embodiments, a solution of pure acetone is used as a fixation reagent. In some embodiments, a solution of pure ethanol is used as a fixation reagent. In some embodiments, a solution of 95% ethanol and 5% glacial acetic acid is used as a fixation reagent. In some embodiments, a solution of 50% methanol and 50% acetone is used as a fixation reagent. In some embodiments, a solution of 50% methanol and 50% ethanol is used as a fixation reagent.

The lymphocytes are typically fixed to a substrate to facilitate handling and visualization. Substrates amenable for use with the subject methods are generally rigid or semi-rigid structures made of transparent material. Such materials include, but are not limited to plastic, glass, and/or quartz. In some embodiments, the substrate is a microscope slide made of any suitable material and having any suitable geometry. For example, the substrate can be a glass microscope slide.

In some embodiments, the length of the substrate ranges in size from about 20 mm, up to about 25 mm, up to about 30 mm, up to about 35 mm, up to about 40 mm, up to about 45 mm, up to about 50 mm, up to about 55 mm, up to about 60 mm, up to about 65 mm, up to about 70 mm, up to about 75 mm, up to about 80 mm. In some embodiments, the width of the substrate ranges in size from about 20 mm, up to about 25 mm, up to about 30 mm, up to about 35 mm, up to about 40 mm, up to about 45 mm, up to about 50 mm, up to about 55 mm, up to about 60 mm, up to about 65 mm, up to about 70 mm, up to about 75 mm, up to about 80 mm. In some embodiments, the thickness of the substrate ranges from about 0.5 mm, up to about 0.8 mm, up to about 1 mm, up to about 1.2 mm, up to about 1.5 mm. In some embodiments, the substrate is a standard microscope slide that is approximately 75 mm in length, 25 mm in width, and 1 mm thick.

Once the lymphocytes have been stained with a DNA binding dye, the labeled mitogen is detected to define the cytoplasm boundary of binucleated lymphocytes. The cytoplasm boundary represents the periphery of the cell where it contacts the outside environment, and detecting the cytoplasm boundary allows cells, and in particular binucleated lymphocytes, to be readily identified. Cytoplasm visualization is useful to identify binucleated lymphocytes by observing their distinctive shape, and encourage low false positive rates because of the relatively clear signal provided by labeled mitogen. The two nuclei in a binucleated lymphocyte should have intact nuclear membranes approximately equal in size, and should either be unconnected or only attached through one or more fine nucleoplasmic bridges.

Identification of binucleated lymphocytes should be carried out at a moderate level of magnification, using a microscope. For example, a magnification of 100 to 400×, or in some embodiments 100 to 200×, should be used to identify binucleated lymphocytes by their labeled cytoplasm boundary. Examples of suitable apparatus for this analysis include flow cytometers, confocal microscopes, and laser scanning microscopes. A large number of binucleated lymphocytes should be identified and analyzed for micronuclei to increase the statistical validity of the results. In some embodiments, at least 100 binucleated lymphocytes are analyzed, in other embodiments 100 to 500 binucleated lymphocytes are analyzed, while in further embodiments at least 500 binucleated lymphocytes are analyzed. Typically, reviewing about 500 cells will identify about 100 binucleated lymphocytes.

Once a binucleated lymphocyte has been identified, the micronuclei in the binucleated lymphocyte should be counted. Microncuclei (MN) are biomarkers for chromosome breakage, and are relatively small pieces of chromosomal material found outside of the two main nuclei in the binucleated lymphocyte. MN originate from either lagging whole chromosomes or acentric chromosome fragments. An image of a binucleated lymphocyte including microncuclei is shown in FIG. 3. Counting the micronuclei in a binucleated lymphocyte is typically carried out at a higher level of magnification using a microscope, although in some embodiments a moderate level of magnification may be suitable for both identifying the binucleated lymphocytes and counting the micronuclei within the binucleated lymphocytes. For example, a magnification of 400 to 1000× can be used, or a magnification of 1000× or more can be used to count the micronuclei within the binucleated lymphocyte. MN are morphologically identical, but are smaller than the main nuclei. They have a diameter from about 1/16^{th} to about ⅓^{rd} of the main diameter of the main nuclei, and are not linked or connected to the main nuclei.

In some embodiments, the method also includes the step of detecting the presence of a nucleoplasmic bridge (NPB) in one or more binucleated lymphocytes. Nucleoplasmic bridges are continuous DNA-containing structures linking the nuclei in a binucleated lymphocyte, as shown in FIG. 3. NPB originate from dicentric chromosomes that may be caused by misrepair of double stranded DNA breaks or telomere end fusions, and are another indication of chromosomal damage. Nucleoplasmic bridges usually do not exceed ¼^{th} of the diameter of the nuclei, and have the same staining characteristics as the nuclei. A binucleated lymphocyte can include both NPBs and MNs. The number of NPB counted in binucleated lymphocytes can be incorporated into the scoring process which is used to determine the level of chromosomal damage.

The total number of MN identified in binucleated lymphocytes are then combined in order to provide an observed micronuclei value. The observed micronuclei value for a subject is then compared with a control micronuclei value in order to determining the level of chromosomal damage in the subject. Control micronuclei value can be obtained from a dose response curve, in which the effect of various doses of radiation or a genotoxic agent on the number of micronuclei formed are compared. Alternately, control values can represent the micronuclei levels present in the subject in a sample obtained before the potential hazardous exposure. Control values for micronuclei levels are also known to those skilled in the art. Control values can vary depending on the age and gender of the subject. In some embodiments, the level of chromosomal damage is also used to characterize the exposure of the subject. For example, the level of chromosomal damage can be used to determine if the subject has been exposed to a 1 to 2 Gy, 2 to 4 Gy, 4 to 6 Gy, 6 to 8 Gy, or greater than 8 Gy dose of radiation.

Any of the steps of the method can be automated using suitably designed equipment. In some embodiments, step of counting the micronuclei in one or more binucleated lymphocytes uses automated analysis of digitized images that makes use of an algorithm for automated image analysis. Other examples of automated equipment include a robotic blood handler, an automated metaphase harvester, an automated metaphase spreader, and an automated slide stainer. Automated image analysis can include metaphase finding and image capture, automation of the dicentric assay, and automated scoring of nuclei. See Martin et al., Radiat. Meas. 42, 1119-1124 (2007) and Friedman, L. I., Severns M. L., Vox Sang 51 Suppl. 1, S57-S62 (1986) for a discussion of automation in blood-based biodosimetry. Automated CBMN using laser scanning has also been described. Francois et al., Biotechniques, 57, 309-12 (2014).

The subject methods may be performed manually by one or more technicians, or may be performed automatically, using automated equipment that is designed and/or programmed to carry out the steps of the subject methods. In some embodiments, the subject methods may be performed through a combination of manual performance by technicians and automated performance by equipment. Furthermore, in some embodiments, blood samples from a plurality of subjects are evaluated using the method. Using automated equipment facilitates analysis of blood samples from a plurality of subjects, but is not necessary.

In some embodiments, the subject methods are entirely performed manually by technicians. In other embodiments, the subject methods are performed entirely by automated equipment that has been designed and/or programmed to carry out the steps of the subject methods in a particular order. In some embodiments, one or more of the steps of the subject methods are performed by technicians, while one or more of the steps of the subject methods are performed by automated equipment that has been designed and/or programmed to carry out the designated steps. Any combination of manual and/or automated performance may be used to carry out the subject methods.

In some embodiments, an algorithm is used to determine the level of chromosomal damage. Recent examples of suitable algorithms include the MN software module integrated in the metaphase finder system MSearch™, developed and commercialized by Metasystems (a manufacturer of microscopic imaging systems) that automatically identifies by morphological criteria BN cells by the occurrence of two adjacent similarly DAPI stained nuclei. In a second step, MN are counted automatically in a circular area defined around the two nuclei of the BN cell. See Schunk et al., Cytogenet. Genome Res. 104, 383-389 (2004) and Varga et al., Mutagenesis 19, 391-397 (2004).

In some embodiments, the method of detecting chromosomal damage in a subject further includes a translocation analysis of lymphocytes from a blood sample from the subject. A recognized drawback of the CBMN assays is that the damage is unstable and therefore is eliminated from the peripheral blood lymphocyte pool at the rate that cell renewal occurs. Therefore, analysis for more persistent types of damage, e.g. stable translocations, is needed to address biological dosimetry for old or long term exposures. Translocation analysis can be carried out using karyotyping, or more preferably analysis by fluorescence in-situ hybridization (FISH). FISH employs specific sequences of DNA which can be used as probes to particular part of the genome and then by attachment of various fluorochromes to highlight or ‘paint’ the regions in different colors. Translocations are seen as colored rearrangements in a fluorescence microscope. See Greulich et al., Mutat. Res. 452 (2000) 73-81.

The present invention is illustrated by the following example. It is to be understood that the particular examples, materials, amounts, and procedures are to be interpreted broadly in accordance with the scope and spirit of the invention as set forth herein.

### Example

### Cytokinesis-Block Micronucleus Protocol

(1) The blood sample is collected using lithium heparin anticoagulant.

(2) Typically 0.5 mL of whole blood is added to 4.5 mL of culture medium (RPMI-1640) supplemented with 10 to 15% heat inactivated fetal calf serum, L-glutamine and antibiotics. 100 μL of phytohaemagglutinin (e.g. PHA-M, Sigma, 25 mg/25 mL H₂O) is added to the culture to give a final concentration of 20 μg/mL.

(3) The blood is cultured in tissue culture flasks at 37° C., 5% CO₂ in a humidified atmosphere.

(4) 20 μL cytochalasin-B (Cyt-B) is added to the culture, at 24 hours post PHA stimulation, to give a final concentration of 6 μg/mL. This is the optimum concentration for accumulating BN cells in whole blood cultures. As Cyt-B is difficult to dissolve in aqueous solution a Cyt-B stock solution should be prepared in dimethylsulphoxide (5 mg Cyt-B in 3.3 mL DMSO) and aliquoted and stored until required at −20° C.

(5) The culture is terminated between 68-72 hours post PHA stimulation. The chosen harvest time should maximize the number of BN cells and minimize the number of mononucleated and multinucleated cells.

(6) The cells are centrifuged gently at 180 g for 10 min and the supernatant culture medium is removed.

(7) The cells are hypotonically treated with 7 mL of cold (4° C.) 0.075M KCl to lyse red blood cells, and centrifuged immediately at 180 g for 10 min.

(8) The supernatant is removed and replaced with 5 mL freshly made fixative consisting of methanol: acetic acid (10:1) diluted 1:1 with Ringer's solution (4.5 g NaCl, 0.21 g KCl, 0.12 g CaCl₂ in 500 mL H₂O). The fixative should be added whilst agitating the cells to prevent clumps forming. The cells are then centrifuged again at 180 g for 10 min.

(9) The cells are washed with two to three further changes of freshly prepared fixative consisting of methanol:acetic acid (10:1), this time without Ringer's solution, until the cell suspension is clear.

(10) After removing the supernatant to 1 cm or less above the cell pellet (depending on pellet size), the cells are resuspended gently, and the suspension is dropped onto clean glass slides and allowed to air dry.

(11) For light microscope analysis cells can be stained in 2-6% Giemsa (e.g. Giemsa's Azur-Eosin-Methylene blue solution, Merck) in HEPES buffer (0.03M; pH 6.5) during 10-20 min in the dark, followed by a quick rinse in distilled H₂O and air dried. For fluorescence microscopy cells can be stained, alternatively, in acridine orange (10 μg/mL in phosphate buffered saline pH 6.9) for 2-3 sec.

The complete disclosure of all patents, patent applications, and publications, and electronically available materials cited herein are incorporated by reference. The foregoing detailed description and examples have been given for clarity of understanding only. No unnecessary limitations are to be understood therefrom. In particular, while theories may be presented describing operation of the invention, the inventors are not bound by theories described herein. The invention is not limited to the exact details shown and described, for variations obvious to one skilled in the art will be included within the invention defined by the claims.

What is claimed is:

1. A method of detecting chromosomal damage in a subject, comprising the steps of:
a) stimulating proliferation of the lymphocytes of a blood sample from a subject by contacting them with a labeled mitogen;
b) growing the stimulated lymphocytes in cell culture;
c) contacting the lymphocytes with a mitotic arrest agent to arrest the lymphocytes as binucleate lymphocytes;
d) staining the lymphocytes with a DNA binding dye;
e) detecting labeled mitogen to define the cytoplasm boundary of binucleated lymphocytes;
f) counting the micronuclei in one or more binucleated lymphocytes to obtain an observed micronuclei value; and
g) determining the level of chromosomal damage in the subject by comparing the observed micronuclei value with a control micronuclei value.

2. The method of claim 1, further comprising the step of obtaining a blood sample from the subject.

3. The method of claim 1, further comprising the step of isolating lymphocytes from the blood sample before contacting them with a labeled mitogen.

4. The method of claim 1, wherein the mitogen is concanavalin A.

5. The method of claim 1, wherein the mitogen is labeled with a fluorescent compound.

6. The method of claim 1, wherein the mitotic arrest agent is cytochalasin D.

7. The method of claim 1, wherein the DNA binding dye is 4′,6-diamidino-2-phenylindole (DAPI).

8. The method of claim 1, wherein the subject is suspected of having been exposed to a hazardous level of radiation.

9. The method of claim 1, wherein the subject is human.

10. The method of claim 1, further comprising the step of fixing the lymphocytes to a substrate before staining the lymphocytes.

11. The method of claim 9, wherein the substrate is a glass microscope slide.

12. The method of claim 1, further comprising the step of detecting the presence of a nucleoplasmic bridge in one or more binucleated lymphocytes.

13. The method of claim 1, wherein blood samples from a plurality of subjects are evaluated using the method.

14. The method of claim 1, wherein one or more of the steps are automated.

15. The method of claim 1, wherein the step of counting the micronuclei in one or more binucleated lymphocytes uses an algorithm for automated image analysis.

16. The method of claim 1, wherein the method of detecting chromosomal damage in a subject further comprises translocation analysis of lymphocytes from a blood sample from the subject.
